(19) United States
(12) Patent Application Publication
(10) Pub. No.: US 2025/0099044 A1
(43) Pub. Date: Mar. 27, 2025

(54) SYSTEM AND METHOD FOR PREDICTION OF INJURIES

(71) Applicant: Brinda Venkat, Pleasanton, CA (US)

(72) Inventor: Brinda Venkat, Pleasanton, CA (US)

(21) Appl. No.: 18/885,668

(22) Filed: Sep. 15, 2024

Related U.S. Application Data
(60) Provisional application No. 63/539,860, filed on Sep. 22, 2023.

(51) Int. Cl. A61B 5/00; A61B 5/11

(52) U.S. Cl. CPC A61B 5/7275; A61B 5/0022; A61B 5/1126; A61B 5/4585; A61B 5/6812; A61B 5/7264; A61B 2562/0219

(57) ABSTRACT

Described herein is a computer implemented method and system of predicting risk of injury to an individual. Integrate a microcontroller with one or more sensors including but not limited to an accelerometer, gyroscope, skin pH sensor, temperature and humidity sensor, and strap it to the individual's body part. Instruct the individual to perform one dynamic movement. Measure linear acceleration along three dimensions using the accelerometer. Collect data from the microcontroller and transmit the data to a predictive AI model. Receive a response from the predictive AI model on the risk of injury.

### CROSS-REFERENCE TO RELATED APPLICATIONS

This patent application claims the benefit of U.S. Provisional Application Ser. No. 63/539,860 filed Sep. 22, 2023, the contents of which are incorporated by reference herein.

### BACKGROUND OF THE INVENTION

The inventive concepts in general relates to a medical system and method and, in particular, to the prediction of injuries.

Certain dynamic movements are a cause of injuries. For example, 70% of ACL injuries occur in non-contact situations. In the United States, ACL injuries to high school students, especially football, lacrosse, volleyball, and basketball players has been on the rise. Micro tears accumulated over a prolonged period of time are being recognized as a likely cause of ACL injuries in non-contact situations. The ACL supports up to 500 lb of weight. The ACL directs the tibia, from the end of the femur, down a specific path, to maintain knee joint stability. The majority of ACL tears happen as a result of dynamic movements such as jumping, landing hard, sudden deceleration, and change in direction. Factors such as the playing surface, the hydration level of the athlete, overexertion, and incorrect form while playing a sport may also play a role in non-contact injuries.

There is a need to understand the forces generated on a person's knee(s) due to dynamic movements to help predict and prevent the likelihood of ACL injuries in non-contact situations. The need exists for a system that can predict injuries caused by such dynamic movements.

### SUMMARY OF THE INVENTION

The inventive concepts overcome the disadvantages of the prior art and fulfills the needs noted above by providing an injury prediction system. Described herein is a computer implemented method and system of predicting the risk of injury from dynamic movement.

Exemplarily, described herein is a computer implemented method and system of predicting the risk of anterior cruciate ligament (ACL) injury of an individual. Integrate a microcontroller with an accelerometer and strap it to the individual's knee. Instruct the individual to perform one dynamic movement. Measure linear acceleration along three dimensions using the accelerometer. Collect data from the microcontroller and transmit the data to a predictive AI model. Receive a response from the predictive AI model on the risk of ACL injury. Exemplarily, an inventive concept includes a system to predict anterior cruciate ligament (ACL) tears in non-contact situations.

The inventive concept also includes a system for implementing the above method(s) in a computer system. In this system, the computer comprises a computer-readable storage medium in which the software implementing the above methods are stored and executed. The system includes, among others, a network controller that is communicatively linked to a network such as a Local Area Network (LAN), Wide Area Network (WAN), Internet, or the like.

Further, the inventive concept includes a system for implementing the above methods using a cloud computing architecture. The software implementing the method(s) is stored in an application server in the cloud, and a plurality of injury prediction systems are communicatively linked to the cloud.

Other features and advantages of the inventive concepts will become apparent from the following description of the invention, which refers to the accompanying drawings.

### BRIEF DESCRIPTION OF THE DRAWINGS

FIG. 1 illustrates an anterior cruciate ligament (ACL) tear;

FIG. 2-3 illustrate free body diagrams of tibial forces on the knee joint in accordance with an embodiment of the inventive concepts;

FIG. 4 illustrates the initial study undertaken to determine risk of ACL injury comparing jumping to walking in accordance with an embodiment of the inventive concepts;

FIG. 5 illustrates a flowchart of the experimental procedure in accordance with an embodiment of the inventive concepts;

FIGS. 6-7 illustrate the results from the experimental procedure in accordance with an embodiment of the inventive concepts;

FIGS. 8-9 illustrate the prediction from pre-trained logistic regression

model and sample output from the model in accordance with an embodiment of the inventive concepts;

FIG. 10 illustrates an exemplary comparison of Group 1, Group 2, and Negative control forces in accordance with an embodiment of the inventive concepts;

FIG. 11 the forces experienced in the X direction by an individual of a certain mass while jumping according to an embodiment of the inventive concepts; and

FIG. 12 illustrates the average force generated on the knee for individuals of different masses in accordance with an embodiment of the inventive concepts.

### DETAILED DESCRIPTION OF THE PREFERRED EMBODIMENTS

Disclosed embodiments relate to an injury prediction system and methods of using the same.

The terminology used herein is for the purpose of describing particular embodiments only and is not intended to limit the invention. As used herein, the singular terms “a,” “an,” and “the” are intended to include the plural forms as well, unless the context clearly indicates otherwise.

The term “cloud computing” is defined as a model for enabling convenient, on-demand network access to a shared pool of configurable computing resources (such as networks, servers, storage, applications, and services) that can be rapidly provisioned and released with minimal management effort or service provider interaction. Also, any system providing access via the Internet to processing power, storage, software or other computing services, often via a web browser.

The term “computer-readable storage medium” or “computer-readable storage media” is intended to include any medium or media capable of storing data in a machine-readable format that can be accessed by a sensing device and capable of converting the data into binary format. Examples include, but not limited to, floppy disk, hard drive, zip disk, tape drive, CD-ROM, CD-R, CD-RW, DVD-ROM, DVD-RW, blu-ray disc, USB flash drive, RAM, ROM, solid state drive, memory stick, multimedia card, CompactFlash, holographic data storage devices, minidisc, semiconductor memory or storage device, or the like.

The term “machine learning” or “ML” is defined as a subfield of artificial intelligence which is broadly defined as the capability of a machine to imitate intelligent human behavior, or the field of study that gives computers the ability to learn without explicitly being programmed.

The term “supervised learning” is defined as a subcategory of machine learning and artificial intelligence and is a machine learning approach defined by its use of labeled datasets to train or supervise algorithms to classify data or predict outcomes accurately. Supervised learning methods may be classification or regression.

The term “unsupervised learning” is defined as a machine learning approach that uses machine learning algorithms to analyze and cluster unlabeled datasets and these algorithms discover hidden patterns in data without the need for human intervention. Unsupervised learning models may use learning techniques such as clustering, association or dimensionality reduction.

The term “labeled dataset” is defined as a designation for pieces of data that have been tagged with one or more labels identifying certain properties or characteristics, or classifications or contained objects.

The term “deep learning” is defined as a type of machine learning based on artificial neural networks in which multiple layers of processing are used to extract progressively higher level features from data.

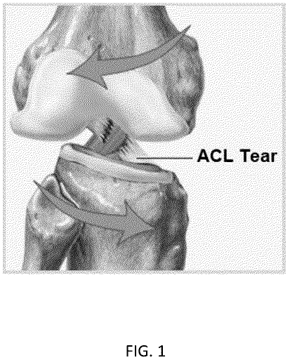

Referring to FIG. 1, an anterior cruciate ligament (ACL) injury is the over-stretching or tearing of the ACL in the knee. A tear may be partial or complete. The knee joint is located where the end of the thigh bone (femur) meets the top of the shin bone (tibia). Four main ligaments connect these two bones: (1) medial collateral ligament (MCL) runs along the inside of the knee and prevents the knee from bending inward; (2) lateral collateral ligament (LCL) runs along the outside of the knee and prevents the knee from bending outward; (3) anterior cruciate ligament (ACL) is in the middle of the knee and prevents the shin bone from sliding out in front of the thigh bone; and (4) posterior cruciate ligament (PCL) works with the ACL and prevents the shin bone from sliding backward under the femur.

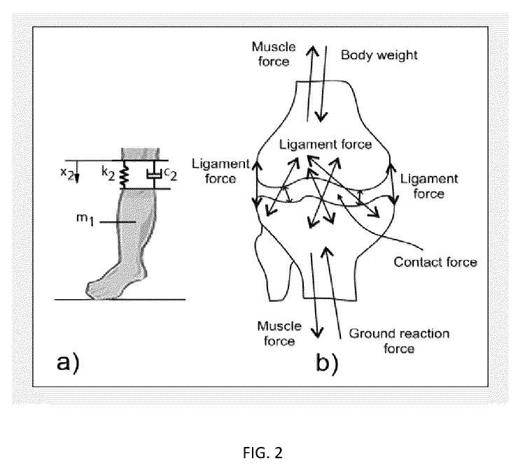

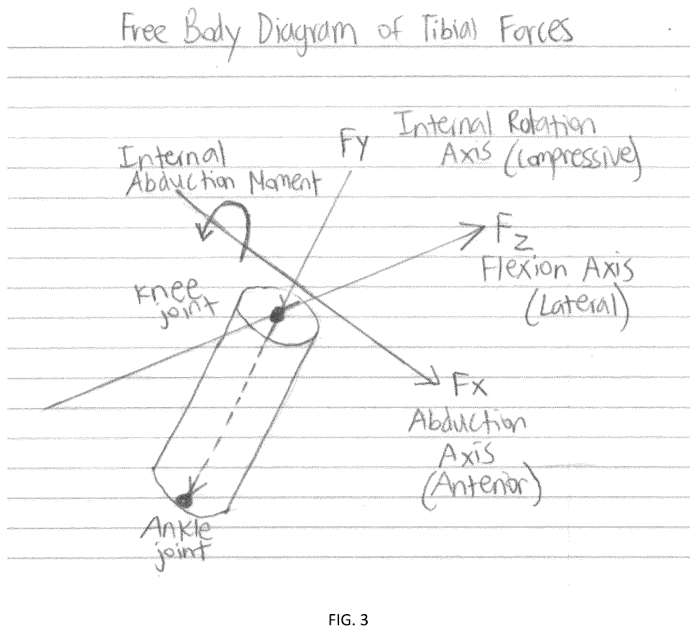

Referring to FIG. 2, the various forces experienced by the knee are as shown. These forces include the body weight, ligament force, contact force etc. Referring to FIG. 3, free body diagram of tibial forces on the knee joint are as shown.

Exemplarily, described herein is a method and system for the prediction of the likelihood of ACL injury based on a person's mass and the linear forces generated on the person's knee while jumping. Repeated overuse and accumulation of micro tears over time could lead to a sudden rupture of the ACL in non-contact situations. Most ACL tears happen as a result of dynamic movements such as jumping, landing hard, sudden deceleration, and change in direction.

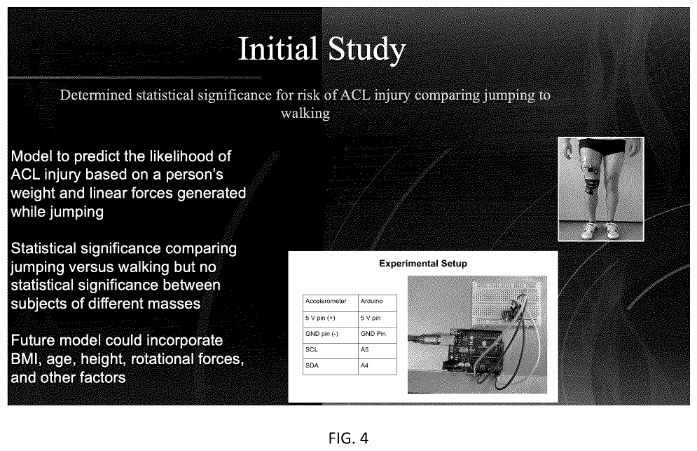

Exemplarily, two individuals of masses 75 kg and 50 kg participated in a study. No statistical significance was found between individual mass and the linear forces generated on the knee. However, there is statistical significance in ACL occurrence when comparing jumping to walking activities. The experimental setup for the study is as shown in FIG. 4.

In one example, the apparatus used to measure and predict ACL tear comprises an accelerometer, a means to attach the accelerometer to a person's leg, and a microcontroller. Exemplarily, the apparatus includes Arduino Uno, Breadboard, Laptop, Wires, SunFounder ADXL345 3-axis accelerometer, knee brace, Velcro, rubber bands, and a 25-ft USB cable.

TABLE 1
Injury Prediction System of the Inventive Concept

| | |
|---|---|
| Micro Controller: | Raspberry Pi4, Intel Edison, Arduino Uno |
| Breadboard: | BB400 Solderless Plug-in |
| Accelerometer: | SunFounder ADXL345 3-axis accelerometer |
| Gyroscope: | HiLetgo GY-521 MPU-6050, SunFounder MPU6050 |
| Temperature Sensor: | SunFounder DS18B20 |
| Humidity Sensor: | SunFounder DHT11 |

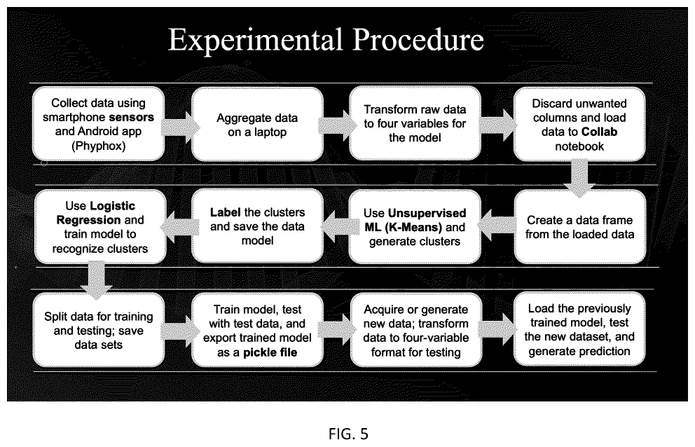

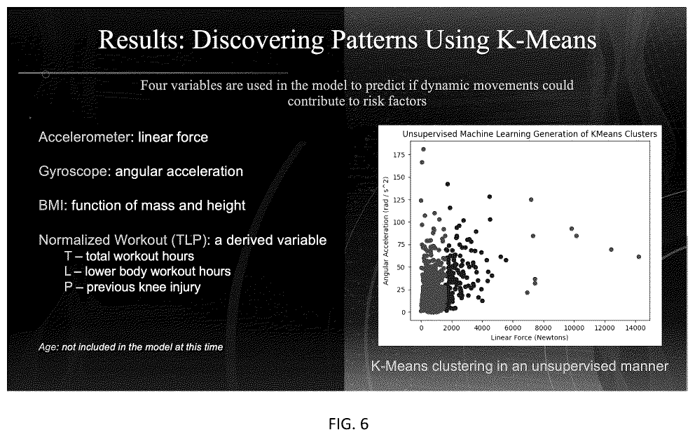

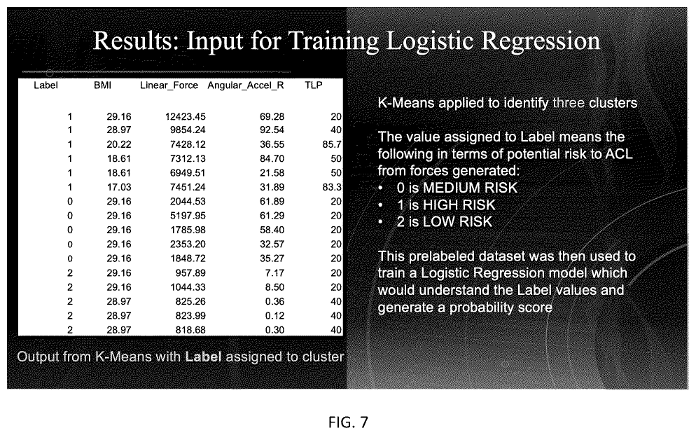

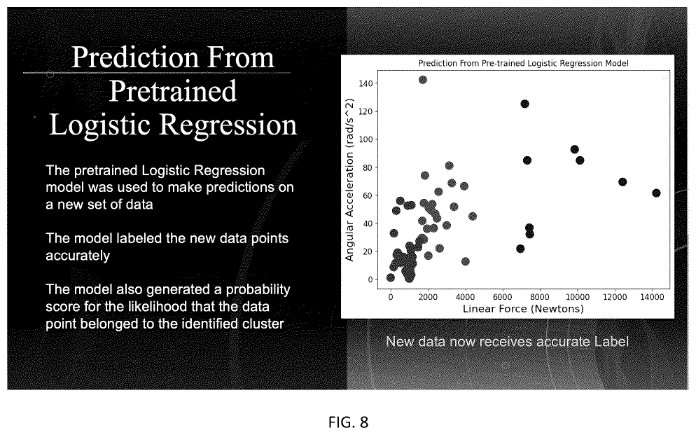

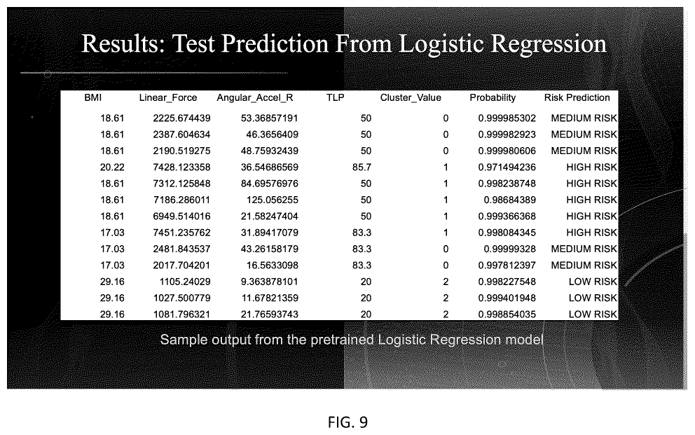

Referring to FIG. 5, exemplarily, described herein is a method and system to predict ACL tears in non-contact situations. Collect data from tibial forces that place the ACL in danger of tearing. Integrate a microcontroller, for example, an Arduino Uno, with an accelerometer and strap it to an individual's knee. The person is instructed to perform one dynamic movement: jump high from a squatting position and land back on their feet. The accelerometer measures linear acceleration along three dimensions. Collect data and transmit it to a predictive AI model for injury predictions and receive a response. For example, after exercise, a user transmits the raw sensor data collected from a wearable, and the predictive AI model provides a prediction of whether they are wearing out their knees and putting themselves at risk of ACL injury. Repeat the above procedure with one other individual. Compute the reaction forces on the knee. Based on the mass of each individual, compute the body weight and the forces generated as a multiple of the body weight. Compare the model from existing data and estimate the occurrence of ACL tears for each person with prolonged exercise. Using Generative AI, the predictive AI model may also recommend improvements to their exercises based on the forces on the knees that were computed from the sensor data that was shared. This would allow someone to change their exercise pattern or approach and be aware of their exercise regimen. There will be different AI models for predicting different kinds of injuries. Also, multiple types of sensors may collect data and send them to the model. If additional sensors are available, certain injury predictions may take into account all such sensor data.

In addition to capturing accelerometer and force related data, additional sensors, such as a gyroscope, temperature sensor, humidity sensor, skin sweat PH sensor to detect hydration, an oxygen pulse oximeter, and others may be installed, and comprehensive data capture may be performed. The predictive AI models used could also be trained with additional features that incorporate the data from additional sensors and values from one or more of these additional sensors into consideration while making the prediction. For example, a module can transform raw sensor data into features such as linear force, rotational force, angle, acceleration, time series data, etc., and can speed up the data collection and transformation process. Standard sensors such as temperature, humidity, Inertial Measurement Units (accelerometer, gyroscope, magnetometer), pH, light sensor, ultrasonic sensor, atmospheric pressure, air quality, oxygen saturation, ECG, EEG, glucose meter, blood pressure, heart rate, and many common sensors capture raw data and need transformation into useful data points before using them in statistical and machine learning applications.

The application contains algorithms and transformation functions to use one or more data streams and transform the raw data to a data format that can be used by the AI model for prediction. Collecting and storing sensor data on the cloud can also help in sharing and aggregating datasets. There are many sources of data in addition to sensors and wearables. The smartphone itself has many sensors. Vendors are also providing modules to extend the smartphone functionality with pluggable hardware and software to expand its functionality. Additional data sources include medical devices, medical imaging systems such as MRIs and X-rays, public domain datasets, external systems, databases, and many more. This data can be collected in real-time, batch, as files, user input through screens, and many other ways. The data can be aggregated through an aggregation engine, transformed, and trained as per rules and algorithms.

Analysis can be conducted on the cleaned data, or the data can be packaged into data sets for further processing. The data can be used as feedback to improve sensor design, combined with social media, and prompts to become part of Large Language Models (LLMs). The data can be queried with prompts and become a part of several use cases in areas such as sports medicine, personal medicine, research, injury prevention, disease identification and diagnosis, telemedicine, patient recovery, and even health insurance. The results of statistical analysis or machine learning and AI analysis can be visualized through dashboards, reports, and charts, or packaged into trained models that can be used by external systems for scoring and prediction.

Described herein are exemplary benefits of the invention. Approximately 70% of current ACL injuries are non-contact based. Many high school athletes, especially female lacrosse and basketball players are at high risk for non-contact ACL injury. If their training data is collected and shared with the PredictiveAI platform, they might be able to get recommendations and predictions on individual athlete risks and this could help the high school staff tailor the exercises to each individual athlete depending on their role in the sport, their height, and weight and the type of forces each athlete generates on their knees while training.

In a pure statistical model, consider the null hypothesis that the mass of a person does not significantly affect the linear forces on the knee and will not result in an increase in the likelihood of ACL tearing. At the same time, statistical significance was seen when comparing the linear forces generated on the knee when jumping compared to walking. Including other characteristics collected from a sensor such as rotational forces on the knee and characteristics that are independent of a sensor such as the playing surface leads to rejecting the null hypothesis and accepting the alternative hypothesis that there is a significance to non-contact ACL injuries. In AI and machine learning models, a risk score can be generated for the provided any input data with a low score indicating a low risk of a non-contact injury and a higher score indicating a higher risk of a non-contact injury, based on the sensor data, and other no-sensor data that is captured and sent to the pre-trained machine learning model. Unsupervised machine learning approaches may be able to associate the data sent to the model to situations that are labeled, for example high risk, medium risk, and low risk. Supervised machine learning approaches may be able to provide a probabilistic score for risk of injury.

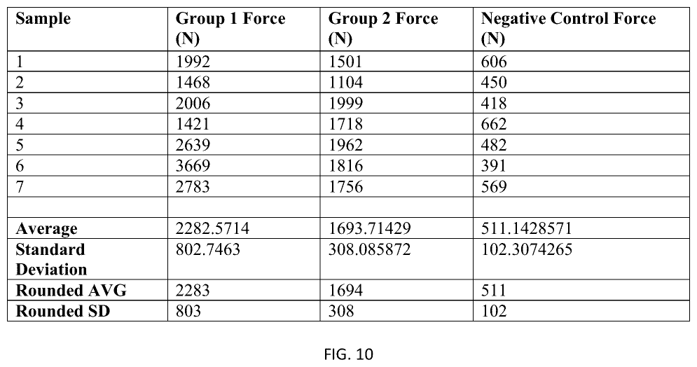

FIG. 10 illustrates an exemplary comparison of Group 1, Group 2, and Negative control forces. The forces represented for Group 1 were 2283±803 N which is comparable to forces for Group 2, which were 1694±308 N. Both groups were significantly different from the Negative Control, which was 511±102 N, as per ANOVA (one-way one way, p-value=1.16677×10−3). The multiple comparison tests did not result in any significance and therefore, we accept the null hypothesis in our results (Data as mean±standard deviation, *=p<0.05 t-test, n=7).

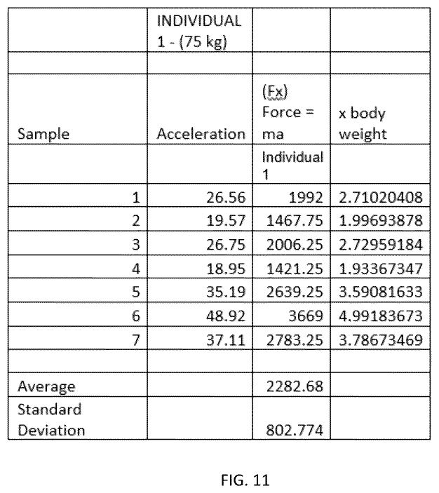

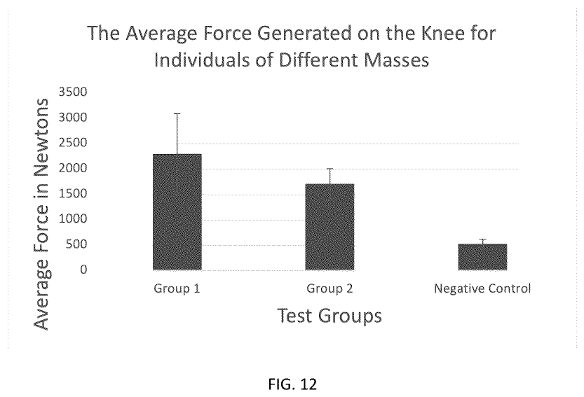

FIG. 11 illustrates the forces experienced in the X direction by an individual of a certain mass while jumping. For example, the force experienced by an individual with a mass of 75 kg for different values of acceleration was measured.

A cloud computing architecture in which methods according to various embodiments of the inventive concepts may be implemented. A plurality of injury prediction system may be communicatively linked to the cloud. An injury prediction application may be hosted on the cloud.

The cloud may be a private cloud, community cloud, combined cloud, hybrid cloud, or any other cloud model. The cloud may have services such as Software as a Service (SaaS), which eliminates the need to install and run an application on a client machine; Platform as a Service (PaaS), which facilitates a computing platform in the cloud; and Infrastructure as a Service (IaaS), which delivers computer infrastructure such as servers, storage and network equipment on the cloud. The cloud may be hosted by any of the public cloud services such as Amazon AWS, Microsoft Azure, Google Cloud, IBM Cloud, Oracle Cloud, or the like.

Alternatively, the network connecting the plurality of injury prediction system may be a Local Area Network (LAN), Wide Area Network (WAN), Internet, an intranet system, an extranet system, or the like. The network may have one of several topologies including, but not limited to, point-to-point, bus, star, ring, tree, mesh and hybrid. The plurality of injury prediction system and the network may be communicatively linked using 100Base-T Ethernet, digital subscriber line (DSL), integrated service digital network (ISDN), DS lines, dedicated T1/T3 lines, fiber-optic cables, satellite dish, wireless, or the like.

The foregoing examples have been provided merely for explanation and are in no way to be construed as limiting the method and system of injury prediction described herein. The method and system described herein can be applied to injury prediction from dynamic movement, and not necessarily restricted to ACL tears. While the method and system has been described with reference to various embodiments, it is understood that the words, that have been used herein, are words of description and illustration, rather than words of limitation. Furthermore, although the method and system described has been described herein with reference to particular means, materials, and embodiments, it is not intended to be limited to the particulars disclosed herein; rather, it extends to all functionally equivalent structures, methods, and uses, such as are within the scope of the appended claims. While multiple embodiments are disclosed, it will be understood by those skilled in the art, having the benefit of the teachings of this specification, that the method and system described herein are capable of modifications and other embodiments may be effected and changes may be made thereto, without departing from the scope and spirit of the solution disclosed herein.

What is claimed as new and desired to be protected by Letters Patent of the United States is:

1. A computer implemented method of predicting risk of injury to an individual, comprising the steps of:
wrapping a wearable to an individual's body part, the wearable having a plurality of sensors and one of the plurality of sensors being an accelerometer and another one of the plurality of sensors being a gyroscope, the plurality of sensors being communicatively linked to a microcontroller;
measuring linear acceleration along three dimensions using the accelerometer when the individual performs one or more dynamic movements;
measuring rotational acceleration along three dimensions using the gyroscope;
collecting data from the microcontroller and transmitting the data to a predictive artificial intelligence (AI) model; and
receiving a predictive response from the predictive AI model on the risk of injury,
wherein the microcontroller is communicatively linked to a computing device having the predictive AI model.

2. The method of claim 1, wherein the plurality of sensors comprises an accelerometer, gyroscope, skin pH sensor, temperature, and humidity sensor.

3. The method of claim 1, wherein the computing device is cloud-based computing.

4. The method of claim 1, wherein the wearable is a knee brace wrapped around the individual's knee to predict anterior cruciate ligament (ACL) tears when the individual performs one or more dynamic movements.

5. The method of claim 4, wherein the AI model predicts the likelihood of ACL tears based on the individual's body weight and linear forces generated around the individual's knee from the one or more dynamic movements.

6. The method of claim 1, wherein the risk of injury is categorized as a low risk, a medium risk or a high risk.
